(19) United States
(12) Patent Application Publication
(10) Pub. No.: US 2013/0178675 A1
(43) Pub. Date: Jul. 11, 2013

(54) Vaporization of Liquid Halogen by Contact with a Preheated Gas

(71) Applicant: MARATHON GTF TECHNOLOGY, LTD., Houston, TX (US)

(72) Inventors: Sabah A. Kurukchi, Houston, TX (US); Anand Moodley, Secunda (ZA); Yijun Liu, Houston, TX (US); Joseph M. Gondolfe, Magnolia, TX (US); John J. Waycuilis, Cypress, TX (US)

(73) Assignee: MARATHON GTF TECHNOLOGY, LTD., Houston, TX (US)

(21) Appl. No.: 13/733,449

(22) Filed: Jan. 3, 2013

Related U.S. Application Data
(60) Provisional application No. 61/584,754, filed on Jan. 9, 2012.

(51) Int. Cl. C07C 1/30

(52) U.S. Cl. CPC C07C 1/30
USPC 585/310; 252/183.13; 422/649

(57) ABSTRACT

A method is provided for vaporizing a liquid elemental halogen. A heating gas is preheated in the absence of halogen to a preheat temperature which results in a preheated heating gas. The preheated heating gas is directly contacted with a feed of a liquid elemental halogen and heats the feed to a vaporizing temperature sufficient to vaporize at least a portion of the feed to a quantity of an elemental halogen vapor. A gas mixture results which includes the heating gas and the quantity of the elemental halogen vapor.

### BACKGROUND OF THE INVENTION

The present invention relates generally to processes and systems for vaporization of a volatile liquid by saturation into a gas stream, and more particularly, to processes and systems for the saturation of a liquid halogen, such as bromine, into a gas, such as a light hydrocarbon gas, to form a homogenous, saturated gas mixture.

Natural gas, a fossil fuel, is primarily composed of methane and other light alkanes and has been discovered in large quantities throughout the world. When compared to other fossil fuels, natural gas is generally a cleaner energy source. For example, crude oil typically contains impurities, such as heavy metals, which are generally not found in natural gas. By way of further example, burning natural gas produces far less carbon dioxide than burning coal. However, challenges are associated with the use of natural gas in place of other fossil fuels. Many locations in which natural gas has been discovered are far away from populated regions and, thus, do not have significant pipeline structure and/or market demand for natural gas. Due to the low density of natural gas, the transportation thereof in gaseous form to more populated regions is expensive. Accordingly, practical and economic limitations exist to the distance over which natural gas may be transported in its gaseous form.

Cryogenic liquefaction of natural gas to form liquefied natural gas (often referred to as “LNG”) is often used to more economically transport natural gas over large distances. However, this LNG process is generally expensive, and there are limited regasification facilities in only a few countries for handling the LNG. Converting natural gas to higher molecular weight hydrocarbon liquids, which are able to be more economically transported due to their higher density and value, can significantly expand the market for natural gas, particularly stranded natural gas produced far from populated regions. While a number of processes for the conversion of natural gas to higher molecular weight hydrocarbon liquids have been developed, these processes have not gained widespread industry acceptance due to their limited commercial viability. Typically, these processes suffer from undesirable energy and/or carbon efficiencies that have limited their use.

One favorable gas-to-liquids process for converting natural gas to higher molecular weight hydrocarbon liquids includes the following steps: (1) bromination of methane to form methyl bromide (CH₃Br) and hydrogen bromide (HBr) by-product, (2) catalytic synthesis or oligomerization of methyl bromide to form higher molecular weight hydrocarbon liquid products, (3) separation of hydrogen bromide by-product from the hydrocarbon liquid products, (4) regeneration of elemental bromine (Br₂) from hydrogen bromide for reuse in step (1), and (5) recovery of the hydrocarbon liquid products.

Bromination is generally a gas phase reaction of a light hydrocarbon gas with gaseous bromine producing a mixture of alkyl bromides. In gas-to-liquid conversion processes such as recited above it is preferable to maintain an excess of the light hydrocarbon gas in the bromination reactor to increase bromine conversion and also to improve the selectivity of the bromination reaction to alkyl monobromides. In any case, the hydrogen bromide by-product of the bromination reaction is typically converted to liquid bromine in the bromine regeneration step by oxidation of the hydrogen bromide by-product. As such, the liquid bromine must be vaporized before it can be fed back to the bromination reactor as a recycle stream.

Vaporization of liquid bromine can be achieved in a conventional shell and tube heat exchanger, such as a kettle reboiler, thermosyphon or falling film evaporator. In a shell and tube heat exchanger, the liquid bromine is vaporized by conveying it through the tubes of the heat exchanger in a first flowpath while a heat transfer medium such as hot steam is circulated through the shell of the heat exchanger in a second flowpath which surrounds the tubes. The second flow path is separated in fluid isolation from the first flowpath by the tube walls which form a solid impermeable heat transfer plate. The heat transfer medium circulating through the shell indirectly heats the liquid bromine passing through the tubes by heat conduction through the tube walls. In particular, the heat transfer medium contacts one side of the tube wall thereby heating the tube wall. The opposite side of the hot tube wall contacts the liquid bromine, thereby heating and vaporizing the liquid bromine.

Shell and tube heat exchangers for this application must be constructed from corrosion resistant materials since bromine is an aggressively corrosive medium and its high corrosiveness is still further increased in the presence of water. Shell and tube heat exchangers for vaporizing bromine commonly employ shells constructed from glass and tubes constructed from high-cost alloys which are extremely resistant to corrosion from the combination of bromine and water. For example, tantalum tubes are essential in the usual case where the bromine in the heat exchanger contains more than 30 ppm water. In contrast, Hastelloy and Monel 400 tubes can be used if the bromine contains less than 30 ppm water and Inconel 600 tubes can be used if the bromine is essentially dry, i.e., contains less than 1 ppm water. In any case, the use of glass and tantalum as materials of construction undesirably imposes upper end size limitations on the heat exchanger so that such heat exchangers are generally only suitable for relatively small applications. Furthermore, the operating pressure for heat exchangers of this construction is undesirably limited to about 10 barg.

It is readily apparent from the foregoing that bromination is an important step in natural gas conversion processes of the type recited above. More generally, halogenation, including bromination, is an important step in the manufacture of many valuable end-products from light hydrocarbon gas feedstocks, including lower alkanes and alkenes such as methane, natural gas and liquefied petroleum gas (“LPG”). Exemplary end-products manufactured from such feedstocks include transportation fuels, chemicals and alcohols. As such, a need exists for an improved method of vaporizing liquid halogens including bromine for use in natural gas conversion processes and, more generally, in end-product manufacturing processes. Specifically, a need exists for such a method which is relatively simple in design and reduces or eliminates the need for expensive metallurgy in the operating units of the vaporization system. A need further exists for such a method which is suitable for large industrial applications.

### BRIEF SUMMARY OF THE INVENTION

The present invention is a process for vaporizing a liquid elemental halogen. In accordance with the present vaporization process a heating gas is preheated in the absence of halogen and preferably also in the absence of water to a preheat temperature. A preheated heating gas results which is directly contacted with a feed of a liquid elemental halogen. The preheated heating gas heats the feed of the liquid elemental halogen to a vaporizing temperature sufficient to vaporize at least a portion of the feed to a quantity of an elemental halogen vapor. A gas mixture results which includes the heating gas and the quantity of the elemental halogen vapor. In accordance with a preferred embodiment the vaporized elemental halogen is saturated in the heating gas of the gas mixture.

A preferred elemental halogen is bromine and a preferred heating gas comprises a hydrocarbon gas. More particularly, a preferred heating gas is a gas having a majority mole fraction of methane. A preferred preheat temperature is less than the halogenation initiation temperature of the elemental halogen and the heating gas. Alternatively, a preferred preheat temperature is greater than or equal to the boiling point of the liquid elemental halogen. In another alternative, a preferred preheat temperature is within a range from about 200° C. to about 300° C. In yet another alternative, a preferred preheat temperature is within a range from about 275° C. to about 300° C. In any case, a preferred heating gas does not substantially react with the elemental halogen when the liquid elemental halogen is heated with the preheated heating gas. Reactivity between the heating gas and elemental halogen is inhibited by the fact that the temperature of the preheated heating gas rapidly drops upon contact with the liquid elemental halogen as the heating gas provides heat for vaporization of the halogen.

In an alternate embodiment of the present characterization of the invention, the feed of the liquid elemental halogen is a first feed, the preheat temperature is a first preheat temperature, the vaporizing temperature is a first vaporizing temperature, the quantity of the elemental halogen vapor is a first quantity and the gas mixture is a first gas mixture having a first halogen concentration. In this embodiment the process further comprises preheating the first gas mixture to a second preheat temperature which results in a first preheated gas mixture. A second feed of the liquid elemental halogen is directly contacted with the first preheated gas mixture. The first preheated gas mixture heats the second feed of the liquid elemental halogen to a vaporizing temperature sufficient to vaporize at least a portion of the second feed of the liquid elemental halogen to a second quantity of the elemental halogen vapor. As a result a second gas mixture is formed which includes the heating gas and the first and second quantities of the elemental halogen vapor. The second gas mixture preferably has a second halogen concentration which is substantially greater than the first halogen concentration and the first and second preheat temperatures are preferably less than or equal to about 300° C. and are more preferably less than or equal to about 200° C.

In another alternate embodiment of the present characterization of the invention, the first preheating and vaporizing steps comprise a first stage of the process and the second preheating and vaporizing steps comprise a second stage of the process. The process further comprises performing a third stage of the process by repeating the preheating and vaporizing steps with the second gas mixture and a third feed of the liquid elemental halogen to produce a third gas mixture.

In yet another alternate embodiment of the present characterization of the invention, a liquid residual portion of the first feed of the liquid elemental halogen remains after vaporizing the portion of the liquid elemental halogen to the first quantity of the elemental halogen vapor. The process further comprises separating the liquid residual portion of the first feed of the liquid elemental halogen from the first quantity of the elemental halogen vapor. In one preferred alternative, the second feed of the liquid elemental halogen includes at least a portion of the liquid residual portion of the first feed of the liquid elemental halogen.

The present invention is alternately characterized as a process for converting a gaseous lower molecular weight alkane to liquid higher molecular weight hydrocarbon. A heating gas is preheated in the absence of halogen to a preheat temperature which results in a preheated heating gas. A feed of a liquid elemental halogen is directly contacted with the preheated heating gas. The preheated heating gas heats the feed of the liquid elemental halogen to a vaporizing temperature sufficient to vaporize at least a portion of the feed of the liquid elemental halogen to an elemental halogen vapor. As a result a gas mixture is formed which includes the heating gas and the elemental halogen vapor. The gas mixture is reacted to form an alkyl halide and the resulting alkyl halide is reacted to form a liquid higher molecular weight hydrocarbon. A preferred elemental halogen is bromine and a preferred alkyl halide is alkyl monobromide. A preferred heating gas comprises a fresh hydrocarbon gas feed and a recycle gas recovered as a gas by-product from reacting the alkyl halide to form the liquid higher molecular weight hydrocarbon.

The present invention is alternately characterized as a system for converting a gaseous lower molecular weight alkane to a liquid higher molecular weight hydrocarbon. The system comprises a preheat heat exchanger, a halogen vaporizing unit, an alkane halogenation reactor, and an alkyl halide conversion reactor. The preheat heat exchanger preheats a heating gas containing a lower molecular weight alkane in the absence of halogen to a preheat temperature which results in a preheated heating gas. The halogen vaporizing unit receives a feed of a liquid elemental halogen and the preheated heating gas from the heat exchanger. The halogen vaporizing unit directly contacts the liquid elemental halogen and the preheated heating gas therein to heat the liquid elemental halogen to a vaporizing temperature sufficient to vaporize at least a portion of the feed of the liquid elemental halogen to an elemental halogen vapor. As a result a gas mixture is formed which includes the heating gas and the elemental halogen vapor. The alkane halogenation reactor reacts the gas mixture therein to form an alkyl halide. The alkyl halide conversion reactor reacts the resulting alkyl halide therein to form a liquid higher molecular weight hydrocarbon, which is preferably an alkylaromatic having a high octane value.

The invention will be further understood from the accompanying drawings and description.

### BRIEF DESCRIPTION OF THE SEVERAL VIEWS OF THE DRAWINGS

The accompanying drawings illustrate certain aspects of the present invention, but should not be viewed as by themselves limiting or defining the invention.

FIG. 1 is a block flow diagram of a prior art system for performing a gas-to-liquids conversion process;

FIG. 2 is a block flow diagram of a system for performing a gas-to-liquids conversion process which incorporates a liquid vaporization process and system of the present invention;

FIG. 3 is a block flow diagram of a multi-stage embodiment of the liquid vaporization system of the present invention;

FIG. 4 is a block flow diagram of a single-stage embodiment of the liquid vaporization system of the present invention; and

FIG. 5 is a block flow diagram of an alternate single-stage embodiment of the liquid vaporization system of the present invention.

### DETAILED DESCRIPTION OF THE INVENTION

The present invention generally relates to the vaporization of a volatile liquid by saturation into a gas stream, thereby transitioning the volatile liquid from the liquid phase to the gas phase. More particularly the invention in its various embodiments is a process and system for vaporizing a liquid elemental halogen by direct contact with a relatively hot preheated gas, generally termed a heating gas. The present vaporization process and system are described below by way of example, wherein the liquid elemental halogen is liquid elemental bromine and the heating gas is a light hydrocarbon gas. However, the present vaporization process and system may be practiced using any number of different halogens and heating gases within the scope of the invention. As such, it is understood that the present vaporization process and system are not specific to any one liquid halogen or any one heating gas.

In accordance with a first specific embodiment of the present vaporization process and system, relatively cooler liquid elemental bromine is mixed with a relatively hotter preheated light hydrocarbon gas which is preferably methane or natural gas typically containing primarily methane as well as some ethane and possibly some higher alkanes. Mixing cooler liquid elemental bromine with the hotter preheated hydrocarbon gas causes direct contact and corresponding heat transfer between them. The sensible heat of the hotter preheated hydrocarbon gas provides sufficient energy to overcome the latent heat of vaporization in part or in whole. At least some, and preferably most, if not substantially all of the cooler liquid elemental bromine is vaporized resulting in a preferably dry, homogeneous gas mixture of bromine vapor and hydrocarbon gas which is the desired output of the vaporization process and system. In accordance with the present embodiment, a sufficient amount of the liquid elemental bromine is preferably vaporized to approach or reach the saturation level of bromine vapor in the hydrocarbon gas which is determined by the operating temperature and pressure of the vaporization process and the system itself.

Substantially any means for mixing and directly contacting the liquid elemental bromine with the hot preheated hydrocarbon gas to vaporize and preferably saturate the liquid bromine in the hydrocarbon gas may have utility in the vaporization process and system of the present invention. Means for directly contacting the liquid elemental bromine and hot preheated hydrocarbon gas and vaporizing the liquid bromine is generally termed a liquid vaporizing unit. Specific exemplary liquid vaporizing units having utility herein include a packed column having either co-current or counter-current gas/liquid flow in which cooler liquid elemental bromine and hotter preheated hydrocarbon gas directly contact one another to heat and vaporize the liquid elemental bromine. Alternatively, the liquid vaporizing unit can comprise: (1) a primary vessel for retaining the liquid elemental bromine, (2) a mechanism for bubbling the hot preheated hydrocarbon gas through the liquid elemental bromine thereby directly contacting the liquid elemental bromine and hot preheated hydrocarbon gas with one another to vaporize the bromine, and (3) a head space or secondary vessel for capturing the resulting homogeneous gas mixture of bromine vapor and hydrocarbon gas produced in the primary vessel.

In any case, the liquid vaporizing unit preferably does not utilize a conventional heat transfer surface to indirectly heat the liquid elemental bromine with a heat transfer medium, such as steam or the like, positioned on the opposite side of the heat transfer surface. Nor, preferably, does the liquid vaporizing unit include any active heating means such as a burner or other active heating element to directly or indirectly heat the liquid elemental bromine in the liquid vaporizing unit. Substantially all of the heat required to vaporize the liquid bromine is preferably provided by direct contact between the liquid bromine and the hot hydrocarbon gas, which has been preheated to a preheat temperature in isolation from the liquid bromine. By way of clarification, the term “indirect heating” is defined herein as a means for heating a fluid, wherein there is no direct contact or fluid communication between the fluid being heated (e.g., liquid elemental bromine) and the heating gas (e.g., a preheated hydrocarbon gas), thereby indirectly transferring heat from the heating gas to the fluid being heated. Conversely, the term “direct heating” is defined herein as a means for heating a fluid, wherein there is direct contact and fluid communication between the fluid being heated and the heating gas, thereby directly transferring heat from the heating gas to the fluid being heated.

Preheating the hydrocarbon gas to the preheat temperature can be achieved in a separate gas preheating unit upstream of the liquid vaporizing unit or in an upstream gas preheating unit which is integral with the liquid vaporizing unit, but which maintains the hydrocarbon gas in isolation from the liquid bromine during the gas preheating step. It is noted that the hydrocarbon gas entering the gas preheating unit is preferably free of water or bromine in either the gas or liquid state. Alternately, water and bromine are, at most, only present in the hydrocarbon gas in very low concentrations. As such the environment within the gas preheating unit is preferably substantially free of water or bromine to reduce the risk or degree of corrosion of the materials of construction in the gas preheating unit.

In accordance with one preferred embodiment, the temperature to which the hydrocarbon gas can be preheated in the gas preheating unit, i.e., the gas preheat temperature, is limited by the halogenation initiation temperature (or bromination initiation temperature in the case where the selected halogen is bromine). The halogenation initiation temperature is the lowest temperature at which substantial reaction between a selected halogen and heating gas occurs (e.g., between bromine and a hydrocarbon gas). This embodiment is particularly preferred when the materials of construction in the liquid vaporizing unit have been selected to withstand operating temperatures which are well in excess of the halogenation initiation temperature. For example, if the liquid vaporizing unit is fabricated from carbon steel having a nickel or nickel alloy cladding, which can operationally withstand temperatures up to about 600° C., the hydrocarbon gas preheat temperature is preferably selected to approach, but not exceed, about 300° C. which approximates the upper limit of the bromination initiation temperature for most hydrocarbon gases. Exemplary nickel alloy cladding materials having utility herein which are able to withstand operating temperatures well in excess of the halogenation initiation temperature include Hastelloy, Monel and Inconel. Although less preferred than the above-recited exemplary materials of construction due to its high cost, tantalum is likewise suitable for construction of the liquid vaporizing unit because it also has the ability to withstand operating temperatures well in excess of the halogenation initiation temperature.

Other alternate materials suitable for construction of the liquid vaporizing unit include carbon steel lined with a fiber-reinforced polymer (FRP) or an unreinforced polymer. Exemplary polymers which may be used as carbon steel liners include polytetrafluoroethylene (PTFE), which is commonly known as Teflon, polyvinylidenefluoride (PVDF), which is commonly known as Kymar, perfluoroalkoxy (PFA), and the like. However, the above-recited polymeric materials of construction are less desirable than the previously recited materials of construction which have a relatively high temperature tolerance because the present polymeric materials typically have a relatively low temperature tolerance and are generally unable to withstand temperatures at or exceeding the halogenation initiation temperature. As such, when these polymeric materials of construction are used, the hydrocarbon gas preheat temperature for the process is limited at its upper end to no greater than about 200° C. or even no greater than about 150° C. which represents the upper operational temperature limit for these polymeric materials.

It is additionally noted that in both embodiments described above, the hydrocarbon gas preheat temperature upper limit may also be dependent to some extent on the operating pressure of the liquid vaporizing unit.

It is likewise apparent that the materials of construction in the liquid vaporizing unit should be selected such that they are able to withstand the actual operating temperature and pressure in the liquid vaporizing unit without significant degradation. Correspondingly, the operating temperature and pressure of the liquid vaporizing unit downstream of the gas preheating unit should be selected such that no significant reaction occurs between the hydrocarbon gas and bromine, whether liquid or vapor, within the liquid vaporizing unit or any fluid conveyance lines thereafter prior to reaching a bromination reactor (if a bromination reactor is positioned downstream of the liquid vaporizing unit).

The present vaporization process and system are preferably practiced in a single stage at a relatively high preheat and vaporization temperature, i.e., preferably between about 200° C. and about 300° C., and more preferably in the higher end of that range. This high-temperature embodiment is enabled using materials in the liquid vaporizing unit described above by way of example which have a relatively high temperature tolerance, i.e., are able to withstand temperatures well in excess of the above-recited high-temperature range. It has been found that single-stage operation of the present high-temperature embodiment of the vaporization process and system is generally sufficient to achieve the desired bromine concentration in the homogeneous gas mixture produced therein.

The vaporization process and system may, nevertheless, alternately be practiced in multiple stages, particularly when the gas preheating and liquid vaporizing steps are at a relatively low preheat and vaporization temperature, i.e., less than about 200° C. or even less than about 150° C. This low-temperature embodiment is preferably employed when the materials used in the liquid vaporizing unit are not able to withstand temperatures beyond the above-recited low-temperature range. It has been found that multi-stage operation of present low-temperature embodiment of the vaporization process and system (as opposed to single-stage operation) is generally necessary to achieve the desired bromine concentration in the homogeneous gas mixture produced therein.

In accordance with the low-temperature multi-stage embodiment, the first stage comprises a first-stage gas preheating step and a first-stage liquid vaporizing step. The first-stage gas preheating step comprises preheating a hydrocarbon gas to a first-stage preheat temperature in a first-stage gas preheating unit. The first-stage liquid vaporizing step comprises conveying a first-stage liquid elemental bromine and the hydrocarbon gas at the first-stage preheat temperature into a first-stage liquid vaporizing unit where the liquid bromine and hydrocarbon gas contact one another. The heat energy of the hydrocarbon gas at the first-stage preheat temperature evaporates at least a portion of the first-stage liquid elemental bromine into the hydrocarbon gas while staying within the temperature and pressure operating limits of the liquid vaporizing unit and its materials of construction. A resulting first-stage gas mixture of bromine vapor and hydrocarbon gas in the first-stage liquid vaporizing unit is characterized by a first-stage bromine concentration. If not all the first-stage liquid elemental bromine is evaporated into the hydrocarbon gas in the first-stage liquid vaporizing unit, a first-stage residual liquid elemental bromine also remains in the first-stage liquid vaporizing unit.

The first-stage gas mixture and first-stage residual liquid elemental bromine, if any, produced in the first-stage liquid vaporizing unit are separated from one another. A second stage is performed by conveying only the first-stage gas mixture to a second-stage gas preheating unit and preheating the first-stage gas mixture therein to a second-stage preheat temperature. The second-stage gas preheating unit and any subsequent stage gas preheating units are preferably heat exchangers which are constructed from highly corrosion-resistant materials such as nickel or nickel alloys, including Hastelloy C or Inconel 600 because the first-stage gas mixture and any subsequent stage gas mixtures include highly-corrosive bromine vapor. Nevertheless, even more highly corrosion-resistant tantalum, which is prohibitively expensive, is not required as a material of construction in the gas preheating units of the present embodiment. Although the first-stage gas mixture and subsequent stage gas mixtures contain bromine vapor, the gas mixtures are preferably substantially free of water and liquid bromine which pose a significantly greater corrosion risk than bromine vapor alone.

In any case, a corresponding second-stage liquid vaporizing step is performed by conveying the first-stage gas mixture at the second-stage preheat temperature and a second-stage liquid elemental bromine, which may include all or a portion of the first-stage residual liquid elemental bromine, into a second-stage liquid vaporizing unit where the liquid bromine and gas mixture contact one another. The heat energy of the first-stage gas mixture at the second-stage preheat temperature evaporates at least a portion of the second-stage liquid elemental bromine into the first-stage gas mixture, thereby producing a second-stage gas mixture of bromine vapor and hydrocarbon gas having a second-stage bromine concentration, which is preferably substantially greater than the first-stage bromine concentration. If not all the second-stage liquid elemental bromine is evaporated into the hydrocarbon gas in the second-stage liquid vaporizing unit, a second-stage residual liquid elemental bromine also remains in the second-stage liquid vaporizing unit.

The second-stage gas mixture and second-stage residual liquid elemental bromine, if any, produced in the second-stage liquid vaporizing unit are separated from one another. As many additional stages as desired may be performed in substantially the same manner as recited above to achieve a final homogeneous gas mixture having a desired final bromine concentration. In many cases, the desired final bromine concentration is preferably the saturation level of bromine in the hydrocarbon gas. In any case, after the final n^{th}-stage liquid vaporizing step, the n^{th}-stage gas mixture of bromine vapor and hydrocarbon gas produced in the n^{th}-stage liquid vaporizing unit has an n^{th}-stage bromine concentration preferably greater than the n^{th}-1-stage bromine concentration.

The present vaporization process and system have specific utility for producing gas mixtures of bromine vapor and hydrocarbon gas which are a useful feed for an alkane bromination reactor used to catalytically convert bromine and hydrocarbon gases to alkyl bromides and hydrogen bromide. Both the above-described single-stage and multi-stage embodiments of the vaporization process and system can be operationally integrated into a comprehensive gas-to-liquids conversion process. Exemplary prior art gas-to-liquids conversion processes, into which the present vaporization process and system can be integrated, are disclosed in each of the following U.S. patent publications: (1) U.S. Pat. No. 7,348,464, issued Mar. 25, 2008; (2) U.S. Patent Application Publication No. 20080275284, published Nov. 6, 2008; and (3) U.S. Patent Application Publication No. 20110015458, published Jan. 20, 2011. The gas-to-liquids conversion processes disclosed in these patent publications are readily modifiable to incorporate the present vaporization process and system therein.

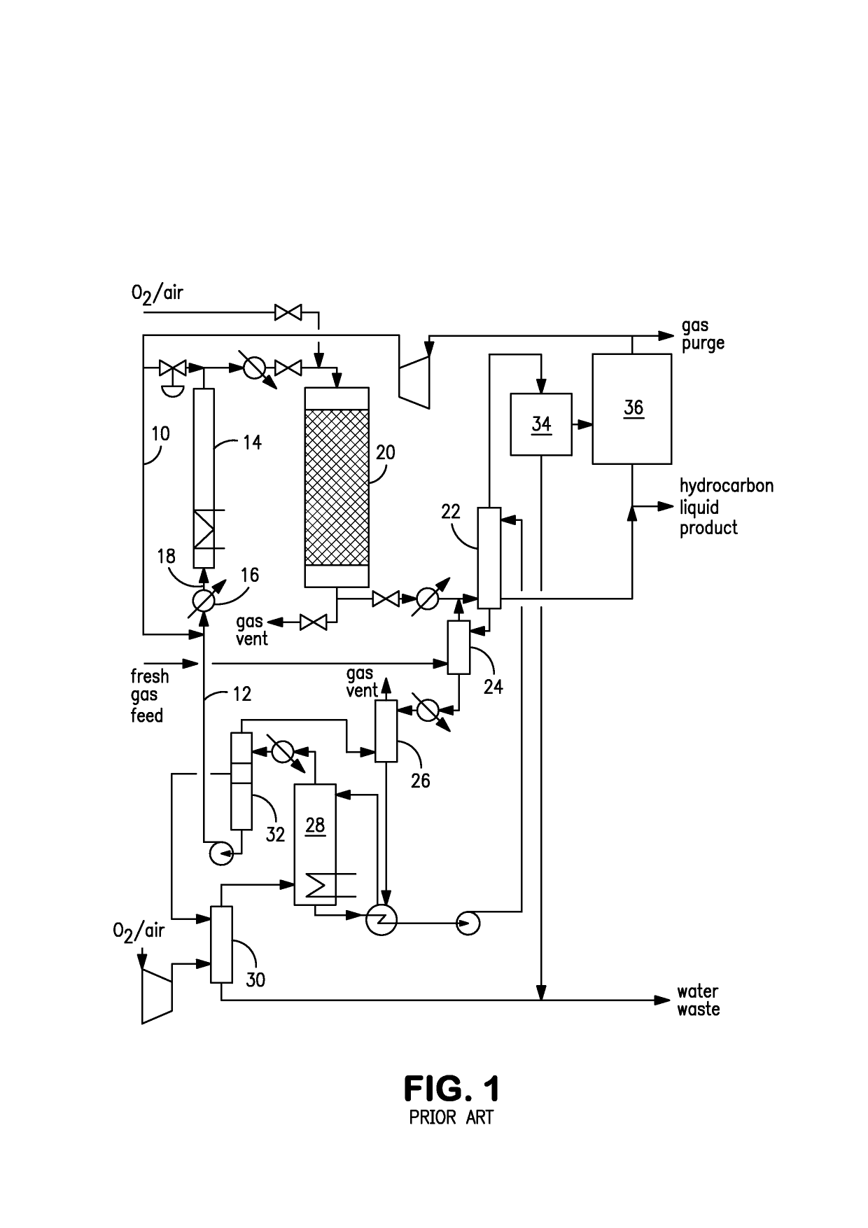

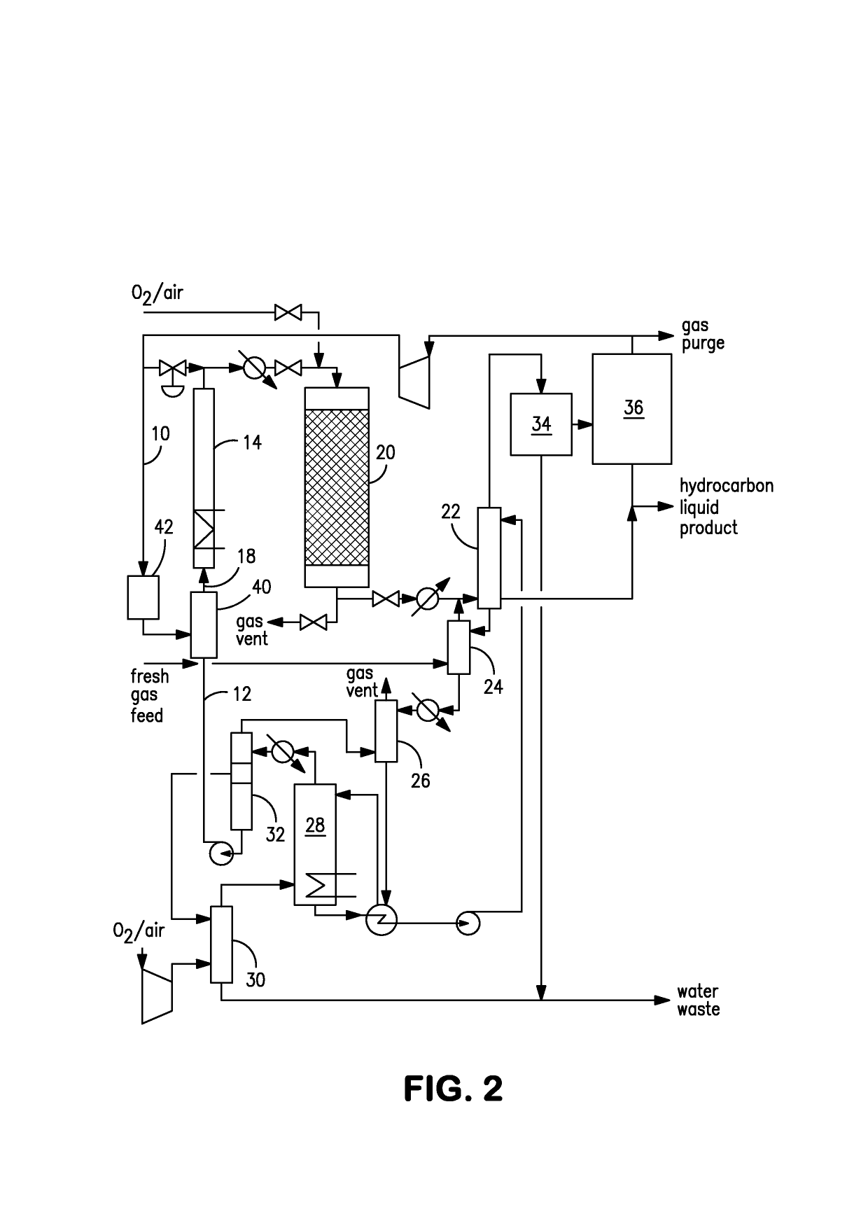

With reference to FIG. 1, an exemplary prior art system is shown schematically for performing a comprehensive gas-to-liquids conversion process. It is noted that FIG. 1 is essentially the same gas-to-liquids conversion system as disclosed in FIG. 2 of U.S. Pat. No. 7,348,464. The present system of FIG. 1 converts gaseous lower molecular weight alkanes to heavier liquid hydrocarbon products by performing multiple functions including bromine vaporization, alkane bromination, alkyl bromide conversion, hydrogen bromide separation, hydrogen bromide oxidation, liquid bromine separation, liquid product dehydration, liquid product separation, gas recycle, liquid product recovery.

The prior art gas-to-liquids conversion process using the system of FIG. 1 is a continuous process which is characterized below starting with the alkane bromination function. A gas return line 10 intersects a liquid return line 12 upstream of an alkane bromination reactor 14 to mix the contents of the two lines therein. The resulting mixture is a two-phase hydrocarbon bromine mixture which includes a partial recycle gas mixture from the gas return line 10 and liquid elemental bromine from the liquid return line 12. Before introducing the two-phase hydrocarbon-bromine mixture into the alkane bromination reactor 14, the two-phase mixture is conveyed through a conventional bromine vaporization heat exchanger 16. The cooler two-phase mixture contacts one side of a heat transfer surface in the heat exchanger 16 while a hotter fluid heat transfer medium such as steam contacts the other side of the heat transfer surface, thereby heating the two-phase mixture in a conventional indirect manner. The two-phase mixture is preferably heated to a temperature greater than the boiling point of elemental bromine at the operating pressure of the heat exchanger 16 which vaporizes essentially all of the elemental bromine in the two-phase hydrocarbon-bromine mixture.

The resulting bromination reactor feed comprising a mixture of the partial recycle gas mixture and bromine vapor exits the heat exchanger 16 and is introduced to the alkane bromination reactor 14 via an alkane bromination reactor inlet line 18. The bromination reactor feed reacts in the alkane bromination reactor 14 to form a bromination reaction product which includes gaseous alkyl bromides and hydrogen bromide vapor. The effluent from the alkane bromination reactor 14, which contains the bromination reaction product, is conveyed to an alkyl bromide conversion reactor 20 and the gaseous alkyl bromides are reacted therein to form higher molecular weight hydrocarbons and additional hydrogen bromide vapor.

The effluent from the alkyl bromide conversion reactor 20, which includes the higher molecular weight hydrocarbons and hydrogen bromide vapor, is fed to a hydrogen bromide scrubber 22 where the effluent is counter-currently contacted with a recirculated aqueous solution likewise fed to the hydrogen bromide scrubber 22. The hydrogen bromide vapor dissolves in the recirculated aqueous solution separating it from the remainder of the effluent. The resulting solution containing the hydrogen bromide vapor is discharged from the bottom of the hydrogen bromide scrubber 22 as a first hydrogen bromide scrubber effluent and fed in series to a hydrocarbon stripper 24, bromine scrubber 26 and hydrogen bromide oxidation reactor 28.

The dissolved hydrogen bromide vapor in the first hydrogen bromide scrubber effluent is either in the form of hydrobromic acid or a metal bromide salt depending on whether the recirculated aqueous solution has neutralized the hydrobromic acid in the hydrogen bromide scrubber 22 or not. Regardless, if the hydrobromic acid is not neutralized to a metal bromide salt in the hydrogen bromide scrubber 22, it is neutralized downstream to form the metal bromide salt in solution before entering hydrogen bromide oxidation reactor 28. The first hydrogen bromide scrubber effluent is modified as it passes through the hydrocarbon stripper 24 and bromine scrubber 26, after which the metal bromide salt solution in the effluent is introduced into the hydrogen bromide oxidation reactor 28. The metal bromide salt is oxidized therein to form elemental bromine by contact with a fresh oxygen or air feed supplied to the hydrogen bromide oxidation reactor 28 from an external source not shown after the oxygen or air feed have been passed through a bromine stripper 30.

A vapor phase mixture containing the elemental bromine is withdrawn from the top of the hydrogen bromide oxidation reactor 28 while the recirculated aqueous solution is withdrawn from the bottom of the hydrogen bromide oxidation reactor 28. The vapor stream from the top of the hydrogen bromide oxidation reactor 28 is cooled and partially condensed resulting in a multi-phase mixture which is separated into three streams in a liquid bromine separator 32. The three streams are a liquid elemental bromine stream, a residual gas stream and a residual water stream. The liquid elemental bromine is discharged from the liquid bromine separator 32 and recycled into the liquid return line 12 to repeat the above-described bromine functions in a new cycle of the gas-to-liquids conversion process. The residual water is passed through the bromine stripper 30 counter-current to the fresh oxygen or air and discharged from the system as a residual water waste stream. The residual gas is passed counter-currently through the bromine scrubber 26 for the removal of any residual bromine and thereafter vented from the system. The recirculated aqueous solution from the hydrogen bromide oxidation reactor 28, which is relatively free of elemental bromine or other bromine constituents, is discharged therefrom and recycled back to the hydrogen bromide scrubber 22 as described above.

A fresh gas feed, which is preferably a methane-rich hydrocarbon gas, is introduced to the system via the hydrocarbon stripper 24 where the fresh gas feed counter-currently contacts the first hydrogen bromide scrubber effluent. The fresh gas feed strips any residual higher molecular weight hydrocarbons from the first hydrogen bromide scrubber effluent in the hydrocarbon stripper 24 and the resulting stripped first hydrogen bromide scrubber effluent is withdrawn from the bottom of the hydrocarbon stripper 24 and conveyed to the bromine scrubber 26. A resulting mixture of fresh gas feed and residual higher molecular weight hydrocarbons is withdrawn from the top of the hydrocarbon stripper 24 and merged with the effluent from the alkyl bromide conversion reactor 20. The merged stream is conveyed to the hydrogen bromide scrubber 22 along with the recirculated aqueous solution from the hydrogen bromide oxidation reactor 28 as described above.

Passing the merged stream through the hydrogen bromide scrubber 22 produces a second hydrocarbon bromide scrubber effluent in addition to the first hydrocarbon bromide scrubber effluent described above. The second hydrocarbon bromide scrubber effluent, which includes the fresh gas feed, water and the bulk of the higher molecular weight hydrocarbons, is discharged from the top of the hydrogen bromide scrubber 22 and conveyed to a product dehydrator 34 where water is separated from the second hydrogen bromide scrubber effluent. The separated water is discharged from the system as a waste with the residual water waste. The remaining dehydrated effluent is conveyed from the product dehydrator 34 to a product separator 36 where the dehydrated effluent is divided by gas-liquid separation into a separated gas stream and a liquid product stream. The liquid product stream comprises essentially liquid higher molecular weight hydrocarbons, which are preferably alkylaromatics having high octane values. The liquid higher molecular weight hydrocarbons are recovered from the system upon discharge from the product separator 36 as the desirable primary end product of the system, namely, hydrocarbon liquid product. In addition, any higher molecular weight hydrocarbons not entrained in either the first or second hydrogen bromide scrubber effluents exiting the hydrogen bromide scrubber 22 are separately retrieved from the hydrogen bromide scrubber 22 and combined with the liquid product stream discharged from the product separator 36 as supplemental hydrocarbon liquid product.

The separated gas stream from the product separator 36 is the partial recycle gas mixture recited above with reference to gas return line 10 which comprises the fresh gas feed and a recycle gas. The recycle gas is essentially any gas in the separated gas stream other than the fresh gas feed. As such, the recycle gas is typically residual by-product gases from the gas-to-liquids conversion process. The partial recycle gas mixture is conveyed back to the alkane bromination stage via the gas return line 10, thereby completing a cycle of the present gas-to-liquids conversion process.

The prior art gas-to-liquids conversion process and system described above and shown in FIG. 1 is readily modified to incorporate the vaporization process and system of the present invention therein as described below with reference to FIG. 2. In particular, the prior art gas-to-liquids conversion system of FIG. 1 is modified by substituting a single-stage liquid vaporizing unit 40, such as one of the above-described embodiments, for the bromine vaporization heat exchanger 16 of FIG. 1. More particularly, the liquid vaporizing unit 40 replaces the bromine vaporization heat exchanger 16 in the liquid return line 12 upstream of the alkane bromination reactor 14.

The prior art gas-to-liquids conversion system is further modified by installing a gas preheating unit 42, such as one of the above-described embodiments, in the gas return line 10 upstream of the liquid vaporizing unit 40. In accordance with the gas-to-liquids conversion process and system of FIG. 2, the heating gas for the vaporization process and system integral therewith is the partial recycle gas mixture, i.e., the combined fresh gas feed and recycle gas, which is conveyed to the gas preheating unit 42 via the gas return line 10. The liquid halogen for the vaporization process and system is the liquid elemental bromine which is conveyed to the liquid vaporizing unit 40 via the liquid return line 12.

The gas-to-liquids conversion process and system of FIG. 2 is practiced by feeding the partial recycle gas mixture to the gas preheating unit 42 via the gas return line 10 and preheating the partial recycle gas mixture therein to the appropriate temperature and pressure conditions. The hot preheated partial recycle gas mixture from the gas preheating unit 42 as well as the liquid elemental bromine in the liquid return line 12 are both fed directly, and preferably separately, into the liquid vaporizing unit 40. The liquid elemental bromine is vaporized in the liquid vaporizing unit 40 by contact with the hot preheated gas. The bromine vapor mixes with the partial recycle gas mixture and the resulting gas mixture, i.e., bromination reactor feed, constitutes the entire feed to the alkane bromination reactor 14. The bromination reactor feed is conveyed from the liquid vaporizing unit 40 and fed to the alkane bromination reactor 14 via the alkane bromination reactor inlet line 18.

The alkane bromination reactor 14 catalytically reacts the bromine vapor and partial recycle gas mixture in the bromination reactor feed to brominate certain hydrocarbon constituents, preferably methane, in the partial recycle gas mixture, thereby forming methyl bromide and hydrogen bromide. The downstream alkyl bromide conversion reactor 20 catalytically converts the methyl bromide to liquid higher molecular weight hydrocarbons which are recovered as hydrocarbon liquid product. The bromine constituents contained in the effluent of the alkyl bromide conversion reactor 20 are separated and processed to recover liquid elemental bromine for recycling to the liquid vaporizing unit 40 via the liquid return line 12 as described above. The bulk of the gas contained in the effluent of the alkyl bromide conversion reactor 20 is separated and combined with the fresh gas feed which jointly form the partial recycle gas mixture. The partial recycle gas mixture is returned to the gas preheating unit 42 via the gas return line 10 in the manner described above.

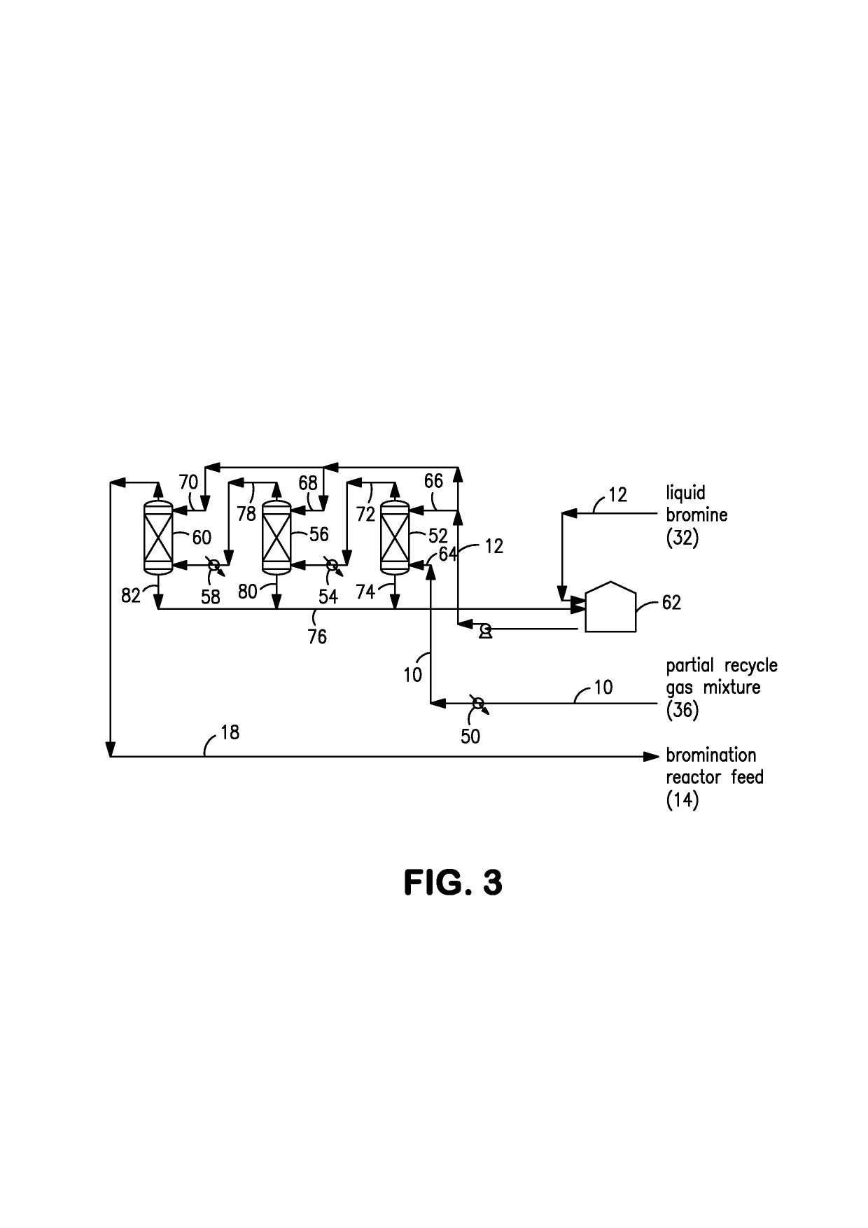

With reference to FIG. 3, a multi-stage embodiment of a vaporization process and system is shown which is capable of replacing the single-stage vaporization process and system integrated into the gas-to-liquids conversion process and system of FIG. 2. The multi-stage vaporization process and system comprise three stages, each including a paired gas preheating step and liquid vaporizing step. The first-stage gas preheating step utilizes a first-stage gas preheating unit 50 which is preferably a heat exchanger. The first-stage liquid vaporizing step which is paired with the first-stage gas preheating step utilizes a first-stage liquid vaporizing unit 52 positioned downstream of the first-stage gas preheating unit 50. The first-stage liquid vaporizing unit 52 is preferably a packed column having counter-current gas/liquid flow.

The second-stage gas preheating step utilizes a second-stage gas preheating unit 54 which is likewise preferably a heat exchanger that is substantially the same or similar to the heat exchanger of the first-stage gas preheating step. The second-stage gas preheating unit 54 is positioned downstream of the first-stage liquid vaporizing unit 52. The second-stage liquid vaporizing step which is paired with the second-stage gas preheating step utilizes a second-stage liquid vaporizing unit 56 is positioned downstream of the second-stage gas preheating unit 54. The second-stage liquid vaporizing unit 56 is preferably a packed column that is substantially the same or similar to the packed column of the first-stage liquid vaporizing step.

The third-stage gas preheating step utilizes a third-stage gas preheating unit 58 which is likewise preferably a heat exchanger that is substantially the same or similar to the heat exchangers of the first-stage and second-stage gas preheating steps. The third-stage gas preheating unit 58 is positioned downstream of the second-stage liquid vaporizing unit 56. The third-stage liquid vaporizing step which is paired with the third-stage gas preheating step utilizes a third-stage liquid vaporizing unit 60 positioned downstream of the third-stage gas preheating unit 58. The third-stage liquid vaporizing unit 60 is preferably a packed column that is substantially the same or similar to the packed columns of the first-stage and second-stage liquid vaporizing steps.

The multi-stage vaporization process and system further comprise a liquid bromine surge tank 62 which can be inserted into the liquid return line 12 in the system of FIG. 2 upstream of the gas preheating and liquid vaporizing stages of FIG. 3. The liquid bromine surge tank 62 receives and stores liquid elemental bromine discharged from the liquid bromine separator 32 of FIG. 2. As such, the liquid bromine surge tank 62 is used to regulate the liquid elemental bromine feed to the first-stage, second-stage and third-stage liquid vaporizing units 52, 56 and 60, respectively.

When the multi-phase vaporization process and system of FIG. 3 is substituted into the gas-to-liquids conversion process and system of FIG. 2, continuous operation is effected by directing the gas return line 10 from the product separator (shown as 36 in FIG. 2) to the first-stage gas preheating unit 50. With continuing reference to FIG. 3, the partial recycle gas mixture is introduced to the first-stage gas preheating unit 50 via the gas return line 10 and a first-stage preheating unit gas inlet line 64. The partial recycle gas mixture is preheated to a first-stage gas preheat temperature in the first-stage gas preheating unit 50 and conveyed to the first-stage liquid vaporizing unit 52 where it is introduced to the bottom of the unit 52.

The liquid return line 12 is directed from the liquid bromine separator (shown as 32 in FIG. 2) to the liquid bromine surge tank 62 and from the liquid bromine surge tank 62 to the respective liquid inlet lines 66, 68, 70 of the first-stage, second-stage and third-stage liquid vaporizing units 52, 56, 60. First-stage dry liquid elemental bromine obtained from the liquid bromine surge tank 62 is introduced to the top of the first-stage liquid vaporizing unit 52 via the first-stage vaporizing unit liquid inlet line 66. A portion of the first-stage dry liquid elemental bromine is vaporized by the preheated partial recycle gas mixture introduced to the bottom of the first-stage liquid vaporizing unit 52 resulting in a first-stage gas mixture comprising bromine vapor and the partial recycle gas mixture. The first-stage gas mixture is characterized by a first-stage bromine concentration.

The first-stage gas mixture is withdrawn from the top of the first-stage liquid vaporizing unit 52 and conveyed to the second-stage gas preheating unit 54 via a first-stage/second-stage gas transfer line 72. A first-stage residual liquid elemental bromine remains in the first-stage liquid vaporizing unit 52 which is withdrawn from the bottom of the unit 52 and returned to the liquid bromine surge tank 62 via a first-stage vaporizing unit liquid outlet line 74 and a common surge tank liquid inlet line 76.

The first-stage gas mixture is introduced to the second-stage gas preheating unit 54 where it is preheated to a second-stage gas preheat temperature and conveyed to the second-stage liquid vaporizing unit 56. The preheated first-stage gas mixture is introduced to the bottom of the second-stage liquid vaporizing unit 56. Second-stage dry liquid elemental bromine obtained from the liquid bromine surge tank 62 is introduced to the top of the second-stage liquid vaporizing unit 56 via the second-stage liquid vaporizing unit inlet line 68. A portion of the second-stage dry liquid elemental bromine is vaporized by the preheated first-stage gas mixture, resulting in a second-stage gas mixture likewise comprising cumulative bromine vapor and the partial recycle gas mixture, but characterized by a second-stage bromine concentration which is greater than the first-stage bromine concentration. The second-stage gas mixture is withdrawn from the top of the second-stage liquid vaporizing unit 56 and conveyed to the third-stage gas preheating unit 58 via a second-stage/third-stage gas transfer line 78. A second-stage residual liquid elemental bromine remains in the second-stage liquid vaporizing unit 56 which is withdrawn from the bottom of the unit 56 and returned to the liquid bromine surge tank 62 via a second-stage vaporizing unit liquid outlet line 80 and the common surge tank liquid inlet line 76.

The second-stage gas mixture is introduced to the third-stage gas preheating unit 58 where it is preheated to a third-stage gas preheat temperature and conveyed to the third-stage liquid vaporizing unit 60. The preheated second-stage gas mixture is introduced to the bottom of the third-stage liquid vaporizing unit 60 and third-stage dry liquid elemental bromine obtained from the liquid bromine surge tank 62 is introduced to the top of the third-stage liquid vaporizing unit 60 via the third-stage liquid vaporizing unit inlet line 70. A portion of the third-stage dry liquid elemental bromine is vaporized by the preheated second-stage gas mixture resulting in a third-stage gas mixture likewise comprising cumulative bromine vapor and the partial recycle gas mixture, but characterized by a third-stage bromine concentration which is greater than the second-stage bromine concentration.

The third-stage gas mixture is withdrawn from the top of the third-stage liquid vaporizing unit 60 and constitutes the bromination reactor feed which is directed to the alkane bromination reactor (shown as 14 in FIG. 2) via the alkane bromination reactor inlet line 18. A third-stage residual liquid elemental bromine remains in the third-stage liquid vaporizing unit 60 which is withdrawn from the bottom of the unit 60 and returned to the liquid bromine surge tank 62 via a third-stage vaporizing unit liquid outlet line 82 and a common surge tank liquid inlet line 72.

The vaporization process and system of the present invention have been characterized above both generally and particularly with respect to different preferred embodiments. The vaporization process and system of the present invention have also been characterized above as a standalone process and system and as an integral subsystem of a gas-to-liquids conversion process and system. An alternate characterization of the vaporization process of the present invention is set forth below which optionally includes a liquid bromine preheating step. A generalized embodiment of the alternate vaporization process comprises the following steps:
(1) preheat a light hydrocarbon gas in a gas preheating unit to a gas preheat temperature which is about 5° C. to 10° C. lower than the bromination initiation temperature that is deemed sufficient to completely vaporize liquid bromine;
(2) directly contact a liquid bromine with the hot preheated light hydrocarbon gas in a liquid vaporizing unit to vaporize the liquid bromine;
(3) if the gas preheat temperature of step (1) is found to be insufficient to completely vaporize the liquid bromine in step (2), preheat the liquid bromine before step (2) in a liquid bromine preheat unit to a temperature no greater than about 10° C. below its bubble point or no greater than the bromination initiation temperature if it is more than 10° C. below the bubble point of the liquid bromine;
(4) if the gas or liquid bromine preheat temperature upper limits are reached in step (1) or (3), combine any un-vaporized liquid bromine from liquid vaporizing unit with the liquid bromine feed and recirculate it back to the liquid vaporizing unit; and
(5) withdraw the resulting saturated homogenous gas mixture of bromine vapor and hydrocarbon gas as an output from the liquid vaporizing unit for downstream uses, such as a feed for a bromination reactor.

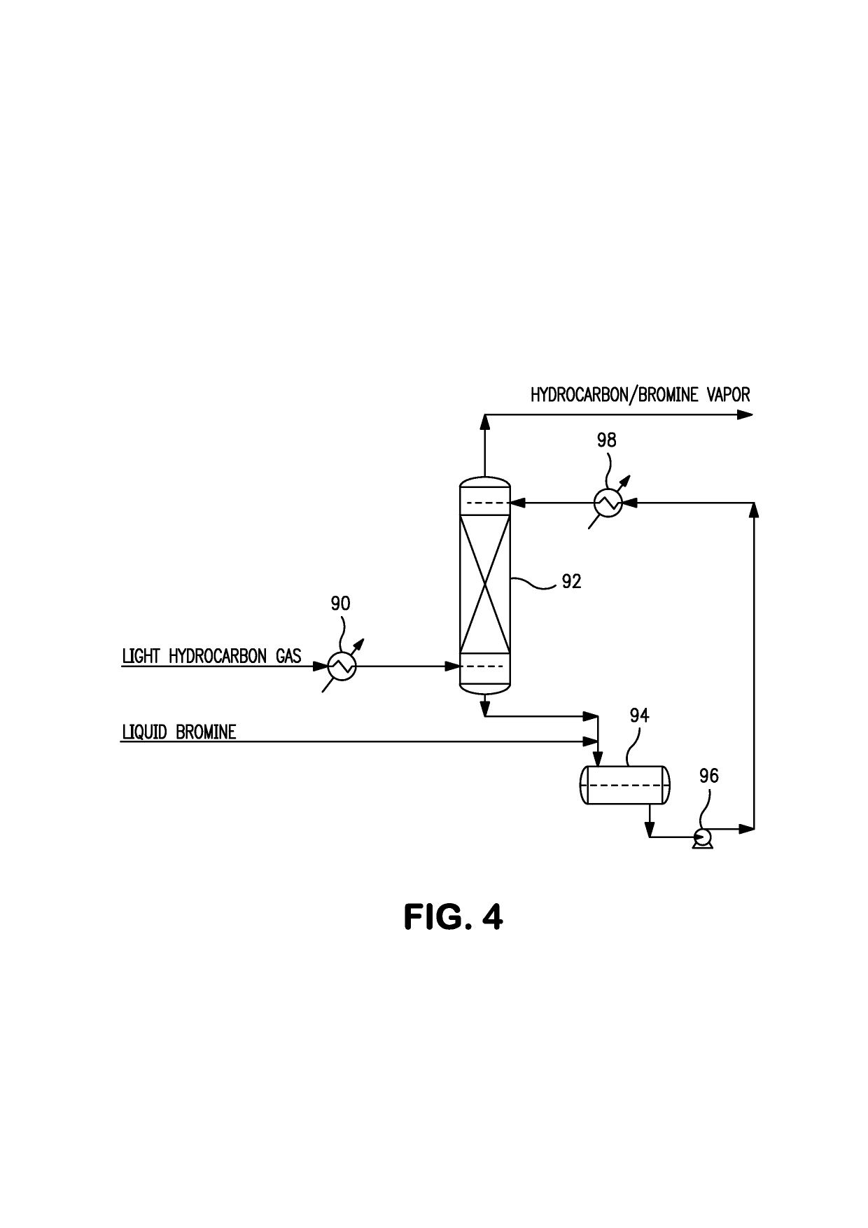

A more specific embodiment of the above-recited vaporization process including the optional liquid bromine preheating step is described further with reference to FIG. 4. A light hydrocarbon gas, i.e., a heating gas, is obtained from an upstream source (not shown) such as a compressor, and preheated in a gas preheating unit 90 which is preferably a conventional carbon steel shell and tube heat exchanger or a fired heater. The resulting hot preheated light hydrocarbon gas is conveyed to a liquid vaporizing unit 92 and introduced into a gas inlet at the bottom of the unit 92, which is preferably a packed column. Liquid bromine is correspondingly obtained from an upstream source (not shown), such as a separation unit or a bromine drying unit, and conveyed to the liquid vaporizing unit 92 where it is introduced into a liquid inlet at the top of the unit 92.

It is noted that reaction of the light hydrocarbon gas and bromine in the liquid vaporizing unit 92 is desirably avoided because it leads to the undesirable selectivity of higher alkyl polybromides over desirable alkyl monobromides. To prevent this reaction, preheating of the light hydrocarbon gas stream in the gas preheating unit 90 is carefully controlled by the practitioner so that the preheat temperature is about 5° C. to 10° C. lower than the bromination initiation temperature. For methane, the bromine initiation temperature is about 270° C. For other light hydrocarbons, such as liquefied petroleum gas (LPG) comprising ethane, propane and butanes, the bromine initiation temperature is reduced to about 230° C. Alkane Bromination Revisited, Lorkovic et al., The Journal of Physical Chemistry A, 2006, v. 110, pp. 8695-8700.

The present embodiment of the vaporization process optionally permits separately preheating the liquid bromine as well as other preliminary steps before the liquid bromine is introduced into the liquid vaporizing unit 92. Election of these optional steps by the practitioner is dependent on the degree of bromine vaporization occurring in the liquid vaporizing unit 92. In particular, if the preheat temperature of the hot preheated light hydrocarbon gas is determined to be sufficient to completely vaporize the liquid bromine in the liquid vaporizing unit 92, an upstream holdup drum (not shown) is provided at the upstream liquid bromine source. The upstream holdup drum serves as a reservoir for the liquid bromine which is conveyed at a required delivery pressure from the upstream holdup drum directly to the liquid inlet of the liquid vaporizing unit 92 as needed by means of a liquid pump and a direct liquid inlet line (not shown).

However, if the gas preheat temperature is determined to be insufficient to completely vaporize the liquid bromine, a downstream holdup drum 94 is provided downstream of the upstream liquid bromine source. The downstream holdup drum 94 likewise serves as a reservoir for the liquid bromine. However, rather than conveying the liquid bromine directly to the liquid inlet of the liquid vaporizing unit 92, the liquid bromine is first conveyed by a liquid pump 96 to a liquid preheating unit 98, such as a conventional shell and tube heat exchanger constructed from high-cost corrosion-tolerant alloys. The liquid bromine is preheated in the liquid bromine preheating unit 98 to a liquid bromine preheat temperature, taking care to maintain the preheat temperature at least 10° C. below the bubble point of the liquid bromine. It is undesirable to vaporize the liquid bromine in the liquid bromine preheating unit 98 because it reduces the heat transfer coefficient and requires heat exchanger equipment which is designed to operate in the two-phase regime. In any case, upon completion of the liquid bromine preheating step, the preheated liquid bromine is conveyed from the liquid bromine preheating unit 98 to the liquid inlet of the liquid vaporizing unit 92.

Regardless of whether the liquid bromine preheating step is elected or not, once the liquid bromine is introduced into the liquid inlet of the liquid vaporizing unit 92 the liquid bromine passes through the top of the packed column in the unit 92 which provides a large contact surface area. As such, the liquid bromine exhibits effective direct contact with the hot preheated light hydrocarbon gas flowing counter-currently up the column packing. The packed column is designed such that substantial vaporization of the bromine occurs across the upper section of the column packing and liquid bromine flow decreases steadily as liquid bromine flows down the column packing reaching essentially zero toward the bottom of the column packing. If minor amounts of liquid bromine slip to the bottom of the packed column during an upset condition or for any other reason, the liquid bromine is discharged from a liquid outlet at the bottom of the packed column to the downstream holdup 94 from which it is recirculated back to the liquid inlet at the top of the liquid vaporizing unit 92.

The desired product of the liquid vaporizing step is a saturated homogenous mixture of bromine vapor and light hydrocarbon gas, which is discharged from a separate gas outlet at the top of the liquid vaporizing unit 92. If the gas line from gas outlet is not well insulated, cooling of the gas mixture will lead to undesirable condensation in the gas line because the gas mixture is at its dew point. One means for preventing condensation is to extend preheating of the hydrocarbon gas or the liquid bromine to ensure that the gas mixture exiting the liquid vaporizing unit 92 is slightly superheated. Another means for preventing condensation is to drop the pressure of the gas mixture exiting the liquid vaporizing unit 92 which effectively superheats the gas mixture. In any case, discharge of the gas mixture from the liquid vaporizing unit 92 completes the present embodiment of the vaporization process.

It is further within the scope of the instant invention to supplement the present vaporization process by routing the gas mixture exiting the gas outlet of the liquid vaporizing unit 92 to an alkane bromination reactor (not shown) as the bromination reactor feed for utilization in a gas-to-liquids conversion process and system in essentially the same manner as described above with respect to previous embodiments. It is noted that hydrocarbon-to-bromine ratios higher than unity are preferred in the alkane bromination reactor of the gas-to-liquids conversion process to ensure both high bromine conversion and high selectivity of the bromination reaction to preferred alkyl monobromides rather than less desirable alkyl polybromides. Higher hydrocarbon-to-bromine ratios also beneficially impact the design of the equipment for the present vaporization process and system. In particular, higher hydrocarbon-to-bromine ratios increase the loading in the gas preheating unit 90, thereby enabling the hydrocarbon gas to provide more sensible heat to the liquid vaporizing step. Accordingly, the need for the liquid bromine stream to provide additional sensible heat to the liquid vaporizing step is reduced, if not eliminated. As a result, the size requirement for the more costly liquid bromine preheating unit 98 upstream of the liquid vaporizing unit 92 is reduced or the liquid bromine preheating unit 98 is eliminated altogether.

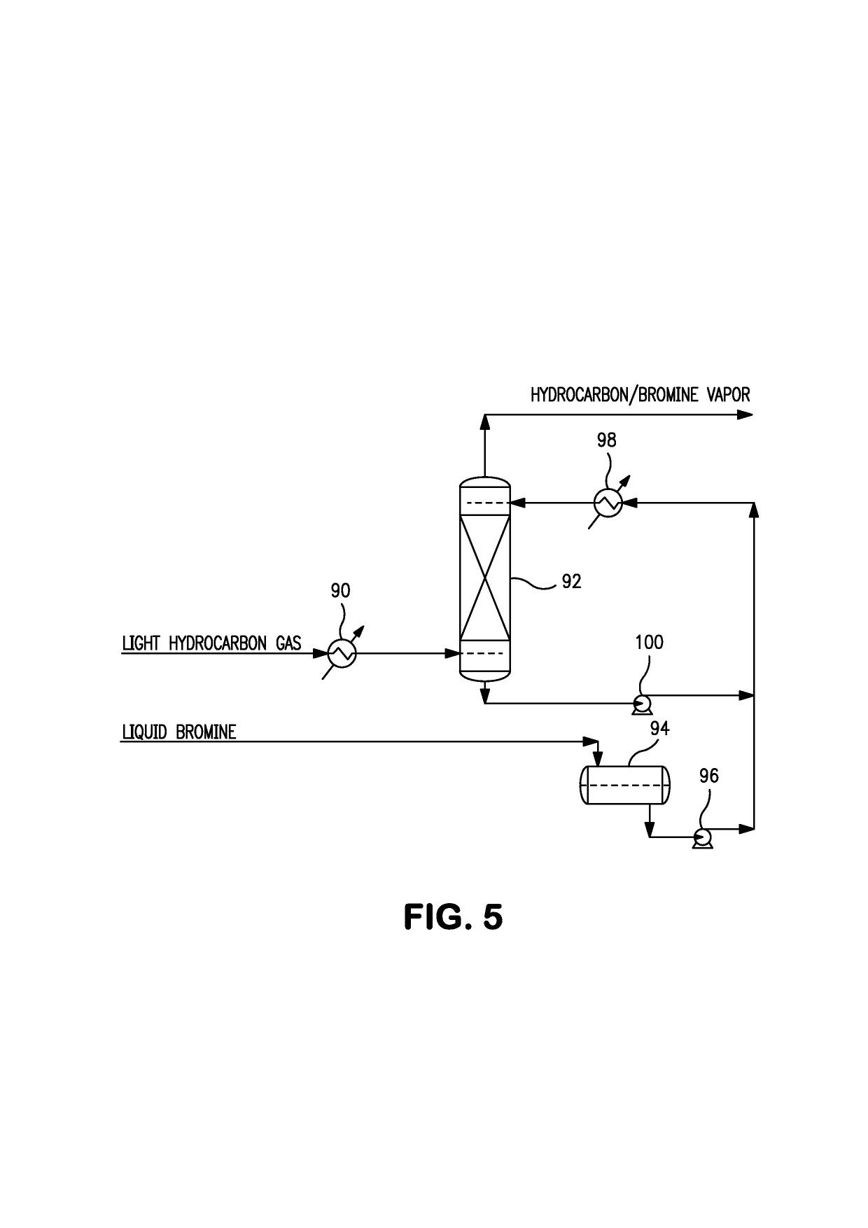

Another specific embodiment of the vaporization process including the optional liquid bromine preheating step is described below with reference to FIG. 5. The elements of FIG. 5 which are common to FIG. 4 are designated in FIG. 5 by the same reference characters used in FIG. 4. The embodiment of FIG. 5 has particular utility at lower hydrocarbon-to-bromine ratios. It is similar to the embodiment of FIG. 4 except that incomplete vaporization of the liquid bromine is permitted in the liquid vaporizing unit 92, i.e., a substantial portion of liquid bromine remains un-vaporized in the liquid vaporizing unit 92. In accordance with this embodiment, the un-vaporized liquid bromine is discharged from the liquid outlet at the bottom of the liquid vaporizing unit 92 and recycled by means of a pump 100 back to the liquid bromine feed line from the downstream holdup drum 94. The resulting combined bromine liquid stream is introduced into the liquid inlet at the top of the liquid vaporizing unit 92. In addition, the present embodiment is preferably performed by preheating both the light hydrocarbon gas and liquid bromine to their respective temperature limits. The bromination initiation temperature is the preheat temperature limit for the light hydrocarbon gas and 10° C. below the bromine bubble point is the preheat temperature limit for the liquid bromine. The specific conditions of the preheating step coupled with the step of recirculating the un-vaporized liquid bromine advantageously ensure complete vaporization of the liquid bromine feed in the vaporization process of the present embodiment.

The vaporization process and system of the present invention provide a number of advantages over prior art vaporization processes and systems. The present vaporization process and system significantly reduce the requirement for expensive metallurgy because cheaper conventional carbon steel heat exchangers can be used for preheating the light hydrocarbon gas rather than more expensive nickel alloys which are required for equipment having direct contact with liquid bromine. In embodiments where the liquid bromine is preheated to supplement the heat input from the preheated gas for bromine vaporization, a less expensive alloy can also be used in the liquid bromine preheating unit than is required for the liquid vaporizing unit.

The present process and system also ensure that the bromination reaction does not occur prior to the alkane bromination reactor by operating below the bromination initiation temperature. The packed column in the liquid vaporizing unit of the present process and system provides a large contact surface area for heat and mass transfer as compared to a trayed column which would require a much larger column for the same function. The counter-current flow in the liquid vaporizing unit also improves efficiency of saturation relative to co-current flow. Furthermore, the homogeneity of the resulting gas mixture discharged from the liquid vaporizing unit as the bromination reactor feed greatly enhances the performance of the alkane bromination reactor.

The following examples demonstrate the scope and utility of the present invention which enable vaporization of liquid elemental bromine. However, these examples are not to be construed as limiting the scope of the present invention.

### Example 1

Natural gas consisting of methane, ethane and propane is preheated at a rate of 262 tons/hour to 170° C. at 6.3 barg pressure. The preheated natural gas is introduced to the bottom of a packed column which is constructed from a carbon steel shell lined with Hastelloy alloy and packed with Hastelloy alloy saddles. Liquid elemental bromine at 54° C. and 6.2 barg is introduced to the top of a packed column at a rate of 392 tons/hour such that the natural gas and liquid bromine flow counter-currently through the packed column. Essentially all of the bromine is vaporized into the gas stream resulting in a gas mixture output flow rate of 654 tons/hour at 55° C. and 6.1 barg. The gas mixture output has a bromine concentration of 59.9 wt % or 13.5 mol %.

### Example 2

The process of Example 1 is repeated except that the inlet pressure to the packed column is 6.8 barg and the outlet temperature is 57° C. The gas mixture exiting the packed column is preheated to 170° C. and introduced into a second-packed column at 6.4 barg. A second-liquid elemental bromine feed is introduced to the top of the second-packed column at a rate of 225 tons/hour and at 54° C. and 6.8 barg. The second-liquid elemental bromine feed is contacted therein with the preheated gas mixture from the first-stage packed column to vaporize the bromine into the gas stream. The resulting gas mixture output from the second-packed column has a bromine concentration of 70.2 wt % or 19.8 mol %. The gas mixture exiting the second-packed column is preheated to 120° C. and introduced into a third-packed column at 6.0 barg. A third-liquid elemental bromine feed is introduced to the top of the third-packed column at a rate of 168 tons/hour and at 54° C. and 6.8 barg. The third-liquid elemental bromine feed is contacted therein with the preheated gas mixture from the second-packed column to vaporize the bromine into the gas stream. The resulting gas mixture is output from the third-packed column at a rate of 1047 tons/hour and at 72° C. and 5.8 barg. The gas mixture output from the third-packed column has a bromine concentration of 75 wt % or 23.8 mol %.

### Example 3

100 tons/hour of dry liquid bromine is vaporized by saturation into 30 tons/hour of pure methane gas employing the flow scheme of FIG. 4 to produce a saturated 3:1 CH₄/Br₂ molar mixture. The latent heat for vaporization of the liquid bromine is inputted to the process as sensible heat by preheating both the methane gas and the liquid bromine. In particular, 1.6 MW of heat is provided to preheat the liquid bromine from 80° C. to 204° C. while ensuring that the liquid bromine is maintained at least 10° C. below its bubble point. 4.4 MW of heat is provided to preheat the methane gas from 80° C. to 270° C. The resulting preheated methane gas is routed to the gas inlet at the bottom of the saturator, while the preheated liquid bromine is routed to the liquid inlet at the top of the saturator. The preheated liquid bromine flows down the packed bed and contacts the hot methane gas flowing upward which vaporizes the bromine. The saturated CH₄/Br₂ mixture is withdrawn from the gas outlet at the top of the packed bed at 30 barg and 128° C. It is noted that by heating the methane gas sufficiently, the saturator can be designed for complete bromine vaporization. The saturator can be designed for an absence of liquid bromine flow through the bottom packing section of the saturator and out the bottom of the saturator.

### Example 4

100 tons/hour of dry liquid bromine is vaporized by saturation into 40 tons/hour of pure methane gas employing the flow scheme of FIG. 4 to produce a 4:1 CH₄/Br₂ molar mixture. The latent heat for vaporization of the liquid bromine is inputted to the process as sensible heat by preheating only the methane gas. In particular, 5.0 MW of heat is provided to preheat the methane gas from 80° C. to 246° C., which is sufficient to ensure complete bromine vaporization in a single saturation stage. The resulting preheated methane gas is routed to the gas inlet at the bottom of the saturator, while the liquid bromine is routed to the liquid inlet at the top of the saturator. The liquid bromine flows down the packed bed and contacts the hot methane gas flowing upward which vaporizes the bromine. A saturated CH₄/Br₂ mixture is withdrawn from the gas outlet at the top of the packed bed at 10 barg and 81° C. It is noted that by using a higher CH₄/Br₂ ratio than Example 4, more sensible heat is added to the methane gas of the present example, thereby obviating the need to preheat the liquid bromine.

### Example 5

The gas feed of the present example is LPG rather than methane gas. As such, the dry liquid bromine is vaporized by saturation into the LPG employing the flow scheme of FIG. 4. 5.3 MW of heat is provided to preheat the LPG from 80 to 230° C., which is sufficient to completely vaporize 100 tons/hour of hot liquid bromine at 164° C. The resulting gas stream exiting the saturator is a saturated gas mixture having 1.8:1 LPG/Br₂ molar ratio.

Table 1 summarizes the results of Examples 3-5, wherein Example 3 is designated Case A, Example 4 is designated Case B and Example 5 is designated Case C.

TABLE 1

| | | Case A | Case B | Case C |
|---|---|---|---|---|
| Hydrocarbon/Br₂ | | 3:1 (C1/Br₂) | 4:1 (C1/Br₂) | 1.8:1 (LPG/Br₂) |
| molar ratio | | | | |
| Light | | | | |
| Hydrocarbon | | | | |
| Gas stream | | | | |
| Flow | t/h | 30 | 40 | 53 |
| Supply | ° C. | 80 | 80 | 80 |
| temperature | | | | |
| Supply pressure | barg | 31 | 11 | 16 |
| Mol Composition | % | CH₄ - 100% | CH₄ - 100% | C₂H₆ - 20% |
| | | | | C₃H₈ - 40% |
| | | | | i-C₄H₁₀ - 20% |
| | | | | n-C₄H₁₀ - 20% |
| Gas Heater Duty | MW | 4.4 | 5.0 | 5.3 |
| Hot gas | ° C. | 270 | 246 | 230 |
| temperature | | | | |
| Br₂ Liquid stream | | | | |
| Flow | t/h | 100 | 100 | 100 |
| Supply | ° C. | 80 | 80 | 80 |
| temperature | | | | |
| Supply pressure | barg | 5 | 5 | 5 |
| Mol Composition | % | Br₂ - 100% | Br₂ - 100% | Br₂ - 100% |
| Br₂ Heater Duty | MW | 1.6 | 0.0 | 1.1 |
| Saturator Br₂ feed | ° C. | 204 | 80 | 164 |
| temperature | | | | |
| Saturator | | | | |
| Bottom liquid | t/h | 0 | 0 | 0 |
| flow | | | | |
| Per pass | | 100% | 100% | 100% |
| vaporization | | | | |
| Exit gas flow | t/h | 130 | 140 | 153 |
| Exit gas | ° C. | 128 | 81 | 119 |
| temperature | | | | |
| Saturator Pressure | barg | 30 | 10 | 15 |

### Example 6

100 tons/hour of dry liquid bromine is vaporized by saturation into 25 tons/hour of pure methane gas employing the flow scheme of FIG. 5 to produce a saturated 2.5:1 CH₄/Br₂ molar mixture. This example is similar to Example 3, but at the lower CH₄/Br₂ ratio of this example there is incomplete vaporization of the liquid bromine. In accordance with this example, 46 tons/hour of liquid bromine is discharged from the liquid outlet at the bottom of the saturator and recirculated to saturator by combining it with the 100 tons/hour liquid bromine feed, preheating the combined liquid stream to 208° C. and feeding the preheated combined liquid stream to the liquid inlet at the top of the saturator. The duty for the gas preheater is reduced by 0.7 MW to 3.7 MW in the present example as compared to Example 3. Correspondingly, the duty for the liquid bromine preheater is increased by 0.7 MW to 2.3 MW. The hot liquid bromine flows down the packed bed of the saturator contacting the hot methane gas flowing up the packed bed, thereby vaporizing 68% of the bromine. The saturated CH₄/Br₂ mixture exits the top of the packed bed at 30 barg and 134° C.

### Example 7

The present example shows that operation at an even lower CH₄/Br₂ molar ratio of 2.0, results in a lower per pass vaporization fraction of 52% and requires increased liquid bromine recirculation of 91 tons/hour.

Table 2 summarizes the results of Examples 6 and 7, wherein Example 6 is designated Case D and Example 7 is designated Case E.

TABLE 2

| | | Case D | Case E |
|---|---|---|---|
| C1/Br₂ molar ratio | | 2.5:1 | 2:1 |
| C1 Gas stream | | | |
| Flow | t/h | 25 | 20 |
| Supply temperature | ° C. | 80 | 80 |
| Supply pressure | barg | 31 | 31 |
| Mol Composition | % | CH₄ - 100% | CH₄ - 100% |
| C1 Heater Duty | MW | 3.7 | 3.0 |
| C1 to Saturator temperature | ° C. | 270 | 270 |
| Supply Br₂ Liquid stream | | | |
| Flow | t/h | 100 | 100 |
| Temperature | ° C. | 80 | 80 |
| Pressure | barg | 5 | 5 |
| Mol Composition | % | Br₂ - 100% | Br₂ - 100% |
| Recycle Br₂ Liquid Stream | | | |
| Flow | t/h | 46 | 91 |
| Temperature | ° C. | 122 | 124 |
| Mixed Br₂ Liquid Stream | | | |
| Flow | t/h | 146 | 191 |
| Temperature | ° C. | 95 | 103 |
| Br₂ Heater Duty | MW | 2.3 | 3.0 |
| Saturator Br₂ feed temperature | ° C. | 208 | 208 |
| Saturator | | | |
| Bottom liquid flow | t/h | 46 | 91 |
| Per pass vaporization | | 68% | 52% |
| Exit gas flow | t/h | 125 | 120 |
| Exit gas temperature | ° C. | 134 | 142 |
| Saturator Pressure | barg | 30 | 30 |

### Example 8

A multi-stage vaporization process and system employing the flow scheme of FIG. 3 are integrated into the gas-to-liquids conversion process and system of FIG. 2. Each stage gas preheating unit of the vaporization system is a heat exchanger employing steam as the heat transfer medium. Each stage liquid vaporizing unit is a counter-current flow packed column.

269 tons/hour of a partial recycle gas mixture containing a dry natural gas feed and a recycle gas at 114° C. and 7.0 barg is fed from the product separator of the gas-to-liquids conversion system to the first-stage gas preheating unit of the vaporization system. The first-stage gas preheating unit provides 10.2 MW of heat to preheat the partial recycle gas mixture which exits the first-stage gas preheating unit at 166° C., 6.9 barg and a rate of 269 tons/hour and is fed to the first-stage liquid vaporizing unit. Liquid elemental bromine from the liquid bromine separator of the gas-to-liquids conversion system is stored in the liquid bromine surge tank of the vaporization system. A first-stage feed of liquid elemental bromine withdrawn from the bromine surge tank is fed to the first-stage liquid vaporizing unit at 54° C., 6.9 barg and a rate of 400 tons/hour and a first-stage portion is vaporized therein. A first-stage gas mixture containing the methane-enrich gas and first-stage portion of bromine vapor is withdrawn from the top of the first-stage liquid vaporizing unit and fed to the second-stage gas preheating unit. A first-stage liquid residual portion of the liquid elemental bromine is withdrawn from the bottom of the first-stage liquid vaporizing unit and returned to the bromine surge tank.

The second-stage gas preheating unit provides 23.2 MW of heat to preheat the first-stage gas mixture which exits the second-stage gas preheating unit at 165° C., 6.6 barg and a rate of 669 tons/hour and is fed to the second-stage liquid vaporizing unit. A second-stage feed of liquid elemental bromine withdrawn from the bromine surge tank is fed to the second-stage liquid vaporizing unit at 54° C., 6.9 barg and a rate of 378 tons/hour and a second-stage portion is vaporized therein. A second-stage gas mixture containing the methane-enrich gas and first-stage and second-stage portions of bromine vapor is withdrawn from the top of the second-stage liquid vaporizing unit and fed to the third-stage gas preheating unit. A second-stage liquid residual portion of the liquid elemental bromine is withdrawn from the bottom of the second-stage liquid vaporizing unit and returned to the bromine surge tank.

The third-stage gas preheating unit provides 0.2 MW of heat to preheat the second-stage gas mixture which exits the third-stage gas preheating unit at 74° C., 6.3 barg and a rate of 1047 tons/hour and is fed to the third-stage liquid vaporizing unit. A third-stage feed of liquid elemental bromine withdrawn from the bromine surge tank is fed to the third-stage liquid vaporizing unit at 54° C., 6.9 barg and a rate of 8 tons/hour and a third-stage portion is vaporized therein. A third-stage gas mixture containing the methane-enrich gas and first-stage, second-stage and third-stage portions of bromine vapor is withdrawn from the top of the third-stage liquid vaporizing unit and third-stage liquid residual portion of the liquid elemental bromine is withdrawn from the bottom of the third-stage liquid vaporizing unit and returned to the bromine surge tank.

The third-stage gas mixture is a bromination reactor feed. The bromination reactor feed is passed through an alkane bromination reactor preheater of the gas-to-liquids conversion system which is a heat exchanger employing the effluent of the alkyl bromide conversion reactor as the heat transfer medium. The alkane bromination reactor preheater provides 31.4 MW of heat to preheat the bromination reactor feed which exits the alkane bromination reactor preheater at 200° C., 6.0 barg and a rate of 1055 tons/hour and is fed directly to the alkane bromination reactor.

While the foregoing preferred embodiments of the invention have been described and shown, it is understood that alternatives and modifications, such as those suggested and others, may be made thereto and fall within the scope of the present invention.

We claim:

1. A process for vaporizing a liquid elemental halogen comprising:
a) preheating a heating gas in the absence of halogen to a preheat temperature, thereby resulting in a preheated heating gas;
b) directly contacting a feed of a liquid elemental halogen with said preheated heating gas; and
c) heating said feed of said liquid elemental halogen with said preheated heating gas to a vaporizing temperature sufficient to vaporize at least a portion of said feed of said liquid elemental halogen to a quantity of an elemental halogen vapor, thereby forming a gas mixture including said heating gas and said quantity of said elemental halogen vapor.

2. The process of claim 1, wherein said heating gas is preheated in the absence of water.

3. The process of claim 1, wherein said elemental halogen is bromine.

4. The process of claim 1, wherein said heating gas comprises a hydrocarbon.

5. The process of claim 1, wherein said heating gas has a majority mole fraction of methane.

6. The process of claim 1, wherein said heating gas does not substantially react with said elemental halogen when heating said liquid elemental halogen with said preheated heating gas.

7. The process of claim 1, wherein said elemental halogen vapor is saturated in said heating of said gas mixture.

8. The process of claim 1, wherein said preheat temperature is less than the halogenation initiation temperature of said elemental halogen and said heating gas.

9. The process of claim 1, wherein said preheat temperature is greater than or equal to the boiling point of said liquid elemental halogen.

10. The process of claim 1, wherein said preheat temperature is within a range from about 200° C. to about 300° C.

11. The process of claim 1, wherein said preheat temperature is within a range from about 275° C. to about 300° C.

12. The process of claim 1, wherein said feed of said liquid elemental halogen is a first feed, said preheat temperature is a first preheat temperature, said vaporizing temperature is a first vaporizing temperature, said quantity of said elemental halogen vapor is a first quantity and said gas mixture is a first gas mixture having a first halogen concentration, and wherein said process further comprises:
d) preheating said first gas mixture to a second preheat temperature, thereby resulting in a first preheated gas mixture;
e) directly contacting a second feed of said liquid elemental halogen with said first preheated gas mixture; and
f) heating said second feed of said liquid elemental halogen with said first preheated gas mixture to a second vaporizing temperature sufficient to vaporize at least a portion of said second feed of said liquid elemental halogen to a second quantity of said elemental halogen vapor, thereby forming a second gas mixture including said heating gas and said first and second quantities of said elemental halogen vapor.

13. The process of claim 12, wherein said second gas mixture has a second halogen concentration substantially greater than said first halogen concentration.

14. The process of claim 12, wherein said first and second preheat temperatures are less than or equal to about 200° C.

15. The process of claim 12, wherein steps a), b) and c) comprise a first stage of said process and steps d), e) and f) comprise a second stage of said process, and wherein said process further comprises performing a third stage of said process by repeating steps d), e) and f) with said second gas mixture and a third feed of said liquid elemental halogen to produce a third gas mixture.

16. The process of claim 12, wherein a liquid residual portion of said first feed of said liquid elemental halogen remains after vaporizing said portion of said liquid elemental halogen to said first quantity of said elemental halogen vapor, and wherein said process further comprises separating said liquid residual portion of said first feed of said liquid elemental halogen from said first quantity of said elemental halogen vapor.

17. The process of claim 16, wherein said second feed of said liquid elemental halogen includes at least a portion of said liquid residual portion of said first feed of said liquid elemental halogen.

18. A process for converting a gaseous lower molecular weight alkane to liquid higher molecular weight hydrocarbon comprising:
preheating a heating gas in the absence of halogen to a preheat temperature, thereby resulting in a preheated heating gas;
directly contacting a feed of a liquid elemental halogen with said preheated heating gas;
heating said feed of said liquid elemental halogen with said preheated heating gas to a vaporizing temperature sufficient to vaporize at least a portion of said feed of said liquid elemental halogen to an elemental halogen vapor, thereby forming a gas mixture including said heating gas and said elemental halogen vapor;
reacting said gas mixture to form an alkyl halide; and
reacting said alkyl halide to form a liquid higher molecular weight hydrocarbon.

19. The process of claim 18, wherein said heating gas is preheated in the absence of water.

20. The process of claim 18, wherein said elemental halogen is bromine.

21. The process of claim 20, wherein said alkyl halide is alkyl bromide.

22. The process of claim 18, wherein said heating gas comprises a hydrocarbon.

23. The process of claim 18, wherein said heating gas has a majority mole fraction of methane.

24. The process of claim 18, wherein said heating gas does not substantially react with said elemental halogen when heating said liquid elemental halogen with said preheated heating gas.

25. The process of claim 18, wherein said elemental halogen vapor is saturated in said heating gas of said gas mixture.

26. The process of claim 18, wherein said preheat temperature is within a range from about 200° C. to about 300° C.

27. The process of claim 18, wherein said preheat temperature is within a range from about 275° C. to about 300° C.

28. The process of claim 18, wherein said heating gas comprises a fresh hydrocarbon gas feed and a recycle gas recovered as a gas by-product from reacting said alkyl halide to form said liquid higher molecular weight hydrocarbon.

29. A system for converting a gaseous lower molecular weight alkane to a liquid higher molecular weight hydrocarbon comprising:
a preheat heat exchanger for preheating a heating gas containing a lower molecular weight alkane in the absence of halogen to a preheat temperature, thereby resulting in a preheated heating gas;
a halogen vaporizing unit for receiving said preheated heating gas from said heat exchanger and a feed of a liquid elemental halogen and directly contacting said liquid elemental halogen and said preheated heating gas to heat said liquid elemental halogen with said preheated heating gas to a vaporizing temperature sufficient to vaporize at least a portion of said feed of said liquid elemental halogen to an elemental halogen vapor, thereby forming a gas mixture including said heating gas and said elemental halogen vapor;
an alkane halogenation reactor for reacting said gas mixture to form an alkyl halide; and
an alkyl halide conversion reactor for reacting said alkyl halide to form a liquid higher molecular weight hydrocarbon.
